(19) United States
(12) Patent Application Publication
(10) Pub. No.: US 2025/0186270 A1
(43) Pub. Date: Jun. 12, 2025

(54) ABSORBENT ARTICLE WITH SHAPING INDICATOR

(71) Applicant: Essity Hygiene and Health Aktiebolag, Göteborg (SE)

(72) Inventor: Angelica BURVALL, Göteborg (SE)

(73) Assignee: Essity Hygiene and Health Aktiebolag, Göteborg (SE)

(21) Appl. No.: 18/859,615

(22) PCT Filed: Jun. 22, 2022
(86) PCT No.: PCT/EP2022/066953

§ 371 Date: Oct. 24, 2024

(51) Int. Cl. A61F 13/472; A61F 13/84

(52) U.S. Cl. CPC A61F 13/472; A61F 13/84; A61F 2013/8497

(57) ABSTRACT

An absorbent article for a female user in the form of a sanitary napkin or incontinence pad including a fluid pervious topsheet, a backsheet and an absorbent core located between the topsheet and the backsheet. The absorbent article includes a shaping indicator, visible from the topsheet, extending along a longitudinal centerline and being symmetric about the longitudinal centerline and asymmetric about a transversal centerline. The shaping indicator has a shaping indicator area, a longitudinal first side edge, a longitudinal second side edge, a transversal front end edge and a transversal rear end edge. The widest transversal width of the shaping indicator is in a front half and the narrowest transversal width of the shaping indicator is in a rear half. The absorbent core includes a core shaping element extending in a longitudinal direction, which in a thickness direction is located within the shaping indicator area.

### TECHNICAL FIELD

The present disclosure pertains to an absorbent article for a female user in the form of a sanitary napkin, incontinence pad or liner having a shaping indicator visual from the topsheet of the absorbent article.

### BACKGROUND

Sanitary napkins and incontinence pads to which this disclosure relates are typically intended to absorb body liquids, such as urine, menses. Such articles may be absorbent pads for heavier bladder leakage, thinner liners for light bladder leakage or sanitary napkins for menses and other body discharges.

Absorbent articles are generally provided with an absorbent core to receive and retain body liquids. For incontinence pads intended for heavier bladder leakage to function efficiently, the absorbent core must quickly acquire body liquids into the structure from the point of application and subsequently distribute the body liquids within and throughout the absorbent core to provide maximum leakage containment. It is also desirable that such pads are comfortable and have a good fit, i.e., to be sized and configured to fit the limited space available in the crotch portion of the underwear while providing the wearer with a feeling of security. Additionally, there is desire that such articles are discreet, thin, and not visible even if the user wears tight fitting which may be seen as being in contradiction with the above objects.

It is furthermore of importance that the absorbent article, such as for example an incontinence pad, gives the user a visual perception of leakage security and good fit, enabling the user to feel secure and thereby enhancing the well-being of the user.

In view of the above, there is a need for an absorbent article for female users, such as for example a sanitary napkin, an incontinence pad or liner, which does not leak, has good fit, is discreet to wear and gives the user a visual perception of leakage security and good fit, enabling the user to feel secure and thereby enhancing the well-being of the user.

### SUMMARY

One or more of the above objects is achieved with an absorbent article according to claim 1. Further advantages and advantageous features of the disclosure are disclosed in the following description and the dependent claims.

An absorbent article for a female user in the form of a sanitary napkin, incontinence pad or liner as disclosed herein has a first and a second longitudinal side edge and a front-end edge and rear end edge. The absorbent article comprises a fluid pervious topsheet, a backsheet and an absorbent core being located between the topsheet and the backsheet. The absorbent article has a longitudinal direction L with a longitudinal centerline Lc, a transverse direction T with a transverse centerline Tc and a thickness direction and has in the longitudinal direction a front half and a rear half. The absorbent article comprises a shaping indicator, visible from the topsheet, extending along a longitudinal centerline Lc and being symmetric about the longitudinal centerline Lc and asymmetric about the transversal centerline Tc. The shaping indicator has a shaping indicator area, a longitudinal first side edge, a longitudinal second side edge, a transversal front end edge and a transversal rear end edge. The widest transversal width of the shaping indicator is in the front half and the narrowest transversal width of the shaping indicator is in the rear half of the absorbent article. The absorbent core comprising a core shaping element extending in the longitudinal direction. The core shaping element is in the thickness direction of the absorbent article located within the shaping indicator area.

The longitudinal first side edge, the longitudinal second side edge, the transversal front end edge and the transversal rear end edge forming a border delimiting the shaping indicator area. The transversal front end edge and the transversal rear end edge of the shaping indicator may be made visual by printing and/or embossing or may be an imaginary line between the front end of the longitudinal first side edge and the front end of the longitudinal second side edge.

The absorbent core is located below the topsheet and comprises a central shaping element extending in the longitudinal direction, optionally along a longitudinal centerline Lc. The central shaping element in the absorbent core is positioned in the thickness direction of the absorbent article and is located within the shaping indicator area. The shaping indicator visualizes the underlying central core shaping element. The core shaping element may be embossed but may also be provided by one or more cut(s), by area(s) with less absorbent material than surrounding area(s) or by areas substantially free from absorbent material.

The shaping indicator may be printed and/or embossed. The shaping indicator may comprise one or more print and/or embossment elements.

The longitudinal first side edge and the longitudinal second side edge of the shaping indicator may comprise a first line and a second line, each extending in the longitudinal direction L on a respective side of the longitudinal centreline Lc. The width of the longitudinal first line and the width of the longitudinal second line of the shaping indicator may vary along the line and the widest width may be in the front half.

The longitudinal first side edge and the longitudinal second side edge of the shaping indicator may diverge in the direction from the rear end edge of the absorbent article towards the front-end edge. The transversal distance between the longitudinal first side edge and the longitudinal second side edge closest to the rear end edge of the article may be 0.5-20 mm and the transversal distance between the first and the second indicator shaping lines closest to the front-end edge may be 40-70 mm.

A circumference around the shaping indicator area may be printed and/or embossed. According to this embodiment he print and/or embossing will be forming a boundary around an un-embossed/not printed shaping indicator area. So, by printing and/or embossing the boundary around the shaping indicator, the un-embossed/not printed shaping indicator area shows a different visual appearance from the printed and/or embossed boundary.

The transversal width of the shaping indicator in the rear half of the absorbent article may be less than 50% of the transversal width of the absorbent article. Optionally the transversal width of the shaping indicator is less than 40%, or less than 30% of the transversal width of the absorbent article.

The disclosed configuration of the absorbent article comprising a core shaping element that adopts and aligns to the female anatomy. The core shaping element allowing the front half of the article to fit over and conform to the flattened forward portion of the female genitalia and to form a front bowl shape for acquiring the body liquids, while the rear half of the article aligns with and fit comfortably into the crease-like regions of the buttocks and perineum.

Furthermore, the disclosed configuration of the absorbent article comprising a shaping indicator supports and gives the user a visual guidance how to wear the absorbent article to closely adopt and align to the female anatomy.

The shaping indicator may be applied at least on the topsheet. According to one embodiment is the shaping indicator printed and is only applied on the topsheet. According to another embodiment the shaping indicator is visualized by both a print on the topsheet and an embossment. The print and/or the embossment may visualize the circumference of the shaping indicator area, or the shaping indicator area.

The longitudinal first side edge of the shaping indicator may be concave as seen from the longitudinal centerline Lc, and the longitudinal second side edge of the shaping indicator may be concave as seen from the longitudinal centerline Lc.

The transversal width of the shaping indicator closest to the rear end edge may be 0.5 to 20 mm and the transversal width of the shaping indicator closest to the front end edge may be 40 to 70 mm.

The absorbent core may have a front part, a rear part and an intermediate part located between the front part and the rear part. The widest transversal width of the absorbent core may be in the front part. The transversal width of the absorbent core may be greater in the entire front part than in the intermediate and rear part. A transition width W₈ between the front part and the intermediate part may be narrower than the transversal width of the rest of the absorbent core. The width of the narrow transversal transition width W₈ may be 50-75% of the widest transversal width of the front part of the absorbent core. The front part of the absorbent core may constitute 15-40% of the longitudinal length of the absorbent core. According to one embodiment is the front part, the rear part and the intermediate part of substantially equal length.

The widest width of the first longitudinal line of the shaping indicator and the second longitudinal line of the shaping indicator may correspond with the transition width W₈ between the front part and the intermediate part of the absorbent core. By having the widest width of the first and second longitudinal lines at the narrow transition width W₈ between the front part and the intermediate part of the absorbent core, the shape of the narrow transition width W₈ of the absorbent core is further visualized by the shaping indicator.

The shaping indicator may be in the form of a Y-shaped or modified Y-shaped indicator comprising a central I-shaped portion provided in the rear half of the absorbent article and a V-shaped or modified V-shaped portion provided in the front half of the absorbent article.

The transversal front-end edge may be concave as seen from the transversal centerline Tc. According to this embodiment the transversal front end has an arc-shape. The arc-shaped front end may be arranged frontwards of the V-shaped or modified V-shaped portion.

The width of the shaping indicator in the rear half may be substantially constant. The maximum width of the shaping indicator in the rear part may be within the range of ±10% of the minimum width of the shaping indicator in the rear half. Optionally, the maximum width of the shaping indicator in the rear part is within the range of ±5% of the minimum width of the shaping indicator in the rear half.

The absorbent article may have an intersection point X_{c} between the longitudinal centerline Lc and the transversal centerline Tc, and an intersection point X_{F1} between a transversal outermost line T_{F} in the front of the shaping indicator and a first longitudinal outermost sideline Lₛ₁. An imaginary line Lα is located between the intersection point X_{c} and the intersection point X_{F1}. An angle a between the imaginary line Lα and the longitudinal centerline Lc may be in the interval between 20° to 45°, optionally between 23° to 30°.

The absorbent core may comprise a first absorbent layer and a second absorbent layer where the first absorbent layer is located between the topsheet and the second absorbent layer, and the second absorbent layer is located between the first absorbent layer and the backsheet. The central embossed shaping element may be in the second absorbent layer. The central embossed shaping element may extend in the longitudinal direction, optionally along a longitudinal centerline Lc.

The second absorbent layer may have a higher basis weight and/or density than the first absorbent layer. The first absorbent layer may have a greater extension than the second absorbent layer. The second absorbent layer may extend over at least part of the front part and the intermediate part of the absorbent core, and the first absorbent layer may extend over the front part, the intermediate part and the rear part of the absorbent core. The rear end of the shaping indicator may end at the transversal end edge of the second absorbent layer.

The second absorbent layer may have a narrowest transversal width corresponding with the transversal transition between the front part and the intermediate part of the absorbent article. The second absorbent layer may comprise a first side cut along a first longitudinal side edge and a second side cut along a second longitudinal side edge. The first side cut is defined between a first front deflection point and a first rear deflection point, and the second side cut is defined between a second front deflection point and a second rear deflection point, and wherein a distance between the front and the rear deflection points of the respective first and second side cuts is within the range of from 20 to 80 mm.

The second absorbent layer may comprise superabsorbent polymers in an amount of from 30 wt. %, 35 wt % or optionally from 40 wt. %. The second absorbent layer may comprise superabsorbent polymers in an amount of from 30 wt. % to 80 wt %.

The first and second longitudinal edges of the absorbent article may be generally straight and essentially parallel to each other. According to this embodiment, the shaping indicator visualize the shape of the absorbent core when the absorbent article has a rectangular shape with generally straight longitudinal edges.

The visual shaping indicator may be printed and/or embossed in a discontinuous pattern. If the visual shaping indicator is printed and/or embossed in a discontinuous pattern the pattern may comprise a few discrete printed and/or embossed elements or a multiply of printed and/or embossed elements/dots.

The absorbent article may also comprise a printed and or embossed longitudinal first contour line(s), visible from the topsheet and extending along the longitudinal first side edge of the article and a longitudinal second contour line(s), visible from the topsheet and extending along the longitudinal second side edge of the article. The contour line(s) are not part of the shaping indicator.

The narrowest transversal width between the longitudinal first contour line and the longitudinal second contour line may correspond with the transition width W₈ between the front part and the intermediate part of the absorbent core. Since the outer contour of the absorbent core may not be visual for a user the shape of the absorbent core may be further visualized by the contour line(s).

The difference between the widest and the narrowest width between the first longitudinal contour line and the second longitudinal contour line may be greater than the difference between the widest and the narrowest width of the absorbent core. This has been seen to enhance the impression of a narrow transition width between the front part and the intermediate part of the absorbent core. The narrowest width between the first and the second longitudinal contour lines may correspond with the transition width W₈ between the front part and the intermediate part of the absorbent article.

The absorbent article may additionally have a rear end direction indicator in the form of a transversal contour rear portion. The part of the end direction indicator may coincide with the longitudinal centre line may have a concave part, as seen from the rear end edge and towards the front-end edge.

The widest width of respective first and the second longitudinal contour lines may correspond with the transition width (W₈) between the front part and the intermediate part of the absorbent core.

The second absorbent layer may extend over at least part of the front part and the intermediate part of the absorbent article, and that the first absorbent layer may extend over the front part, the intermediate part and the rear part of the absorbent article.

The rear end of the shaping indicator may end at the transversal rear end edge of the second absorbent layer.

### BRIEF DESCRIPTION OF THE DRAWINGS

The present invention will be further explained hereinafter by means of non-limiting examples and with reference to the appended drawings wherein:

FIG. 1 a shows a top plan view of an incontinence pad as disclosed herein;

FIG. 1 b shows a top plan view of the incontinence pad as disclosed in FIG. 1 a with an alternative shaping indicator.

FIG. 1 c shows a top plan view of the incontinence pad as disclosed in FIG. 1 a with an alternative shaping indicator.

FIG. 2 shows an exploded view of the incontinence pad in FIG. 1; and

FIG. 3 shows the incontinence pad from FIG. 1, flexed and shaped as during use.

### DETAILED DESCRIPTION

The invention will be described more closely below by reference to an exemplary embodiment. The invention may however be embodied in many different forms and should not be construed as limited to the embodiments set forth in the drawings and the description thereto.

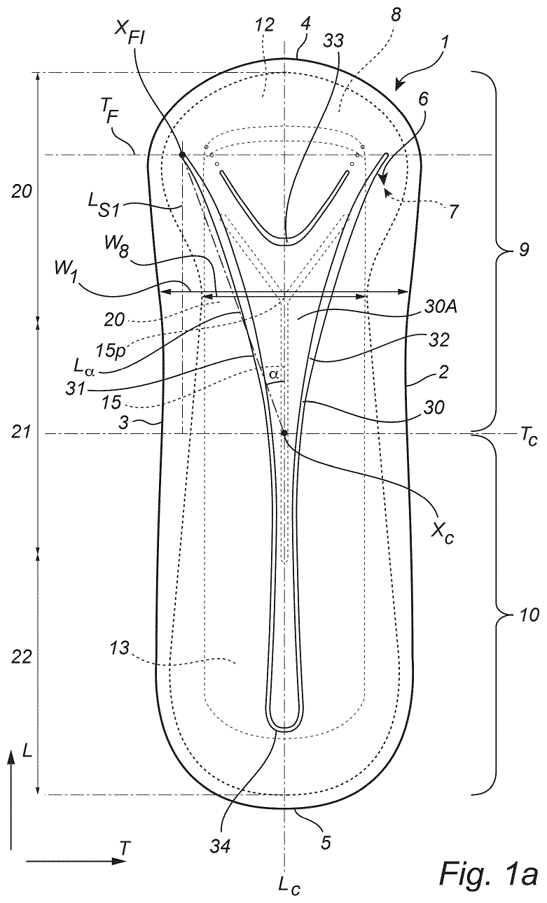

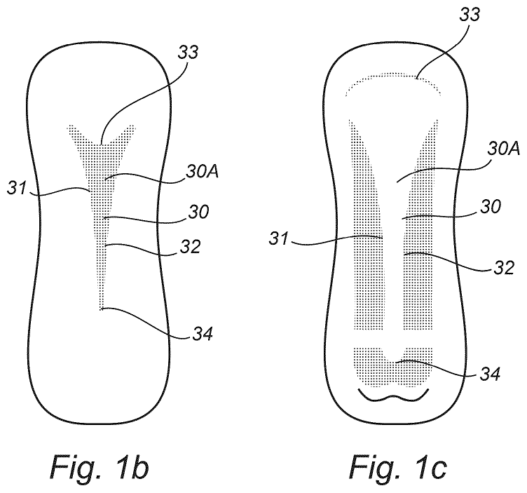

FIGS. 1 to 3 show an incontinence pad 1 according to the present disclosure. FIG. 1 schematically shows the incontinence pad 1 seen from the side that is intended to be facing towards a wearer's body when the pad 1 is being worn.

The incontinence pad 1 has a first and a second longitudinal side edge 2, 3 and a front and a rear end edge 4, 5. The pad 1 comprises a fluid permeable topsheet 6, a backsheet 7 and an absorbent core 8 being located between the topsheet 6 and the backsheet 7. The incontinence pad 1 extends in a longitudinal direction L with a longitudinal centerline Lc, and in a transverse direction T with a transverse centerline Tc and the absorbent core 8 is divided in a front half 9 and a rear half 10. The incontinence pad 1 has a shaping indicator 30, visible from the topsheet 6, extending along a longitudinal centerline Lc and being symmetric about the longitudinal centerline Lc and asymmetric about the transversal centerline Tc. The shaping indicator 30 has a shaping indicator area 30A and a longitudinal first side edge 31 and a longitudinal second side edge 32. The widest transversal width of the shaping indicator 30 is in the front half 9 and the narrowest transversal width of the shaping indicator 30 is in the rear half 10 of the incontinence pad 1.

The absorbent core 8 comprises a first absorbent layer 12 and a second absorbent layer 13. The first absorbent layer 12 being arranged between the topsheet 6 and the second absorbent layer 13.

The topsheet 6 may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The topsheet 6 may consist of non-absorbent fibers, such as thermoplastic fibers. The materials suited as topshet material should be soft and non-irritating to the skin and be readily penetrated by body fluid, such as menstrual fluid and urine.

The backsheet 7 may comprise or consist of a thin plastic film, e.g., a polyethylene or polypropylene film, a nonwoven material coated with a liquid impervious material, a hydrophobic nonwoven, which resist liquid penetration. Laminates of plastic films and nonwoven materials may also be used. The backsheet material can be breathable to allow vapor to escape from the absorbent structure, while still preventing liquids from passing through the backsheet material.

The absorbent core 8 constitutes the absorbent structure of the article which acquires, distributes and stores bodily fluids and comprises the first absorbent layer and the second absorbent layer. The first and second absorbent layers may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form suitable for use in absorbent composites including particles, fibres, flakes, spheres, and the like. A high absorption capacity is provided using high amounts of superabsorbent material. Thin absorbent cores which are common in for example sanitary napkins, baby diapers and incontinence guards, often comprise a compressed, mixed or layered structure of cellulosic fluff pulp and superabsorbent polymers. The size and absorbent capacity of the absorbent core may be varied to suit different product types, such as smaller incontinence pads for day use or larger and longer ones for night-time use.

The first absorbent layer 12 may more specifically be an airlaid nonwoven layer or a matformed absorbent layer.

The second absorbent layer 13 may comprise superabsorbent polymers in an amount of from 30 wt. %, such as 40 wt. % or 50 wt. % or more, optionally within the range of from 30% to 70%. The superabsorbent polymer may for example be mixed with cellulosic fluff pulp. The second absorbent layer 13 is optionally wrapped in a core wrap.

The absorbent core 8 may also comprise a fluid control layer arranged between the topsheet 6 and the first absorbent layer 12. A fluid control layer 14 is adapted to quickly receive and temporarily store discharged liquid before it is absorbed by the first and second absorbent layers 12, 13. A fluid control layer may be a fibrous layer comprising or consisting of synthetic fibers and may for example be airlaid nonwoven, spunlace nonwoven or high loft nonwoven.

The second absorbent layer comprises a shaping element 15 located in the shaping indicator area 30A and extending in the longitudinal direction L along a longitudinal centerline Lc. Hence, the underlying shaping element is located within the shaping indicator area. The first and the second longitudinal print lines of the shaping indicator, enclose the central shaping element and function so as to visualize the underlying shaping element 15.

In FIG. 1a, the underlying central shaping element 15 in the second absorbent layer 13 is an embossed shaping element 15 in the form of a Y-shaped embossing comprising a V-shaped portion 15a provided in the front region 9 of the absorbent core 8 and an I-shaped portion 15b provided at least partly in the rear half 10 of the absorbent core. The V-shaped portion 15a including a V-shaped portion first leg 15c and a V-shaped portion second leg 15d converging within the front part of the absorbent core 8. The I-shaped portion may be a continuous or a discontinuous I-shaped portion. The underlying central embossed shaping element 15 may constitute of a continuous embossing or by discontinuous elements, such as dots, forming the central shaping element 15. The underlying central shaping element 15 may also be provided by one or more cut(s) or by area(s) with less or no absorbent material than surrounding area(s).

The central embossed shaping element 15 may alternatively be a modified Y-shaped portion comprising a modified V-shaped portion, such as with a slightly rounded tip portion or with the V-shaped portion first leg and second leg being non-intersecting.

The shaping indicator 30 provided on the topsheet 6 comprises a first printed line 31 and a second printed line 32, each extending in the longitudinal direction L and on a respective side of the underlying shaping element 15, as seen in the transverse direction T. The first and the second printed lines 31, 32 of the shaping indicator 30 converge towards the rear end edge 5 of the pad 1.

The first and second printed lines 31, 32 are provided with a distance to each other in the rear half 10, or in the rear part 22, as measured in the transverse direction T, being within the range of from 5 mm to 15 mm.

In the FIGS. 1-3, the shaping indicator 30 further also has a transversal printed front end edge 33 and a transversal printed rear end edge 34. The transversal printed front end 33 has an arc-shaped front portion 33 arranged frontwards of the Y shaped underlying shaping element 15 and substantially following the contour of the V-shaped or modified V-shaped portion 15a of the underlying shaping element 15.

The first and the second printed lines 31, 32 of the shaping indicator 30 extend on a respective side of a longitudinal centerline Lc of the pad 1 and wherein a distance d between the first and the second printed lines 31, 32 in a rear third of the absorbent core 8 is within the range of from 0.5 to 20 mm.

The longitudinal first side edge 31 and the longitudinal second side edge 32 of the shaping indicator 30 diverge in the direction from the rear end edge 4 towards the front-end edge 5 of the incontinence pad 1.

The longitudinal first side edge 31 of the shaping indicator is concave as seen from the longitudinal centerline (Lc), and the longitudinal second side edge 32 of the shaping indicator 30 is concave as seen from the longitudinal centerline (Lc).

The absorbent core 8 has a front part 20, a rear part 22 and an intermediate part 21 located between the front part 20 and the rear part 22. The widest transversal width of the absorbent core 8 is in the front part 20.

The incontinence pad 1 has an intersection point X_{c} between the longitudinal centerline Lc and the transversal centerline Tc, and an intersection point X_{F1} between a transversal outermost line T_{F} in the front of the shaping indicator and a first longitudinal outermost sideline Lₛ₁. An imaginary line Lα is located between the intersection X_{c} and the intersection X_{F1} and an angle α between the imaginary line Lα and the longitudinal centerline Lc is in the interval between 20° to 45°, optionally between 23° to 30°.

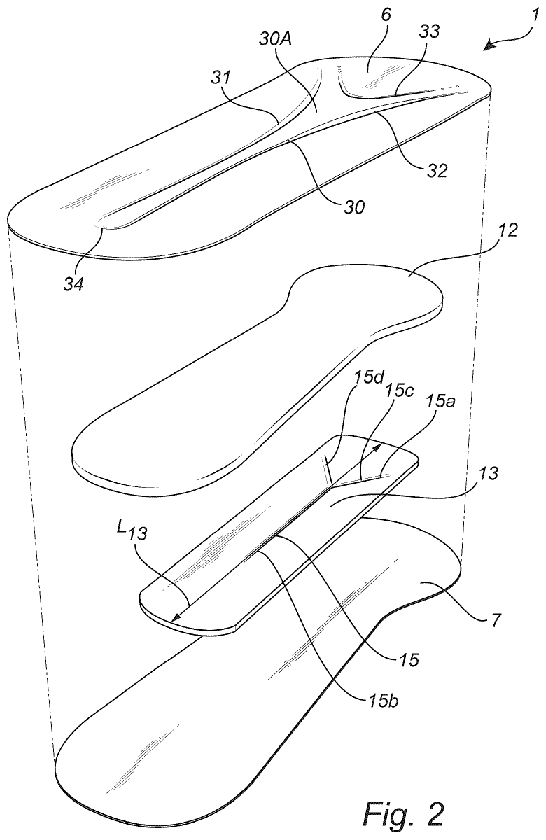

The width of the second absorbent layer 13 for the incontinence pad 1 in FIG. 1-2 is relatively constant, and the maximum width within the rear half of the second absorbent layer 13 is within the range of ±10% of a minimum width of a rear half of the second absorbent layer 13.

The widest transversal width of the shaping indicator 30 is in the front half 9 and the narrowest transversal width of the shaping indicator 30 is in the rear half 10 of the incontinence pad 1. Also, the widest width of the first print line 31, and the widest width of the second print line 32 is in the front half 9. By having a wider width of the print lines 31, 32 in the front half 9, and a widest width of the print lines 31, 32 that also correspond with the transition width (Wt) between the front part and the intermediate part of the incontinence pad 1, where the transversal width of the absorbent core is narrowest, the print lines 31, 32 will enhance the user's visual impression of a narrow core width, even if the core is not visible for the user.

The rear end 34 of the shaping indicator 30 ends at the transversal rear end edge of the second absorbent layer 13.

The difference between the widest and the narrowest width between the first longitudinal shaping element print line and the second longitudinal print line in the front half 9 is greater than the difference between the widest and the narrowest width of the absorbent core 8 in the front half 9. This has been seen to enhance the impression of a narrow transition width between the front part 20 and the intermediate part 21 of the absorbent core 8.

FIG. 1 b shows a top plan view of the incontinence pad 1 as disclosed in FIG. 1 a with an alternative visualization of the shaping indicator 30. In FIG. 1 b is the shaping indicator area 30A printed with a multiply of dots. So, the print pattern comprising a multiply of dots visualizes the shaping indicator 30. FIG. 1b shows an alternative embodiment of the shaping indicator on the incontinence pad 1 a. A circumference around the shaping indicator area 30A is printed. According to an alternative embodiment the circumference around the shaping indicator area 30A is embossed or is formed by a combination of print and emboss. The print in FIG. 1 b forming a boundary delimiting the shaping indicator 30, and the shaping indicator 30 is un-not printed or embossed. So, by printing and/or embossing the boundary around the shaping indicator, the shaping indicator shows a different visual appearance from the printed and or embossed boundary.

FIG. 1 c shows a top plan view of the incontinence pad 1 as disclosed in FIG. 1 a with a further alternative visualization of the shaping indicator 30. In FIG. 1 c is the shaping indicator area 30A not printed but the area around the shaping area 30A is printed. So, the print pattern around the shaping indicator area 30A visualizes the shaping indicator 30. The incontinence pad 1 in FIG. 1 c also has a rear end direction indicator in the form of a transversal contour rear portion. The part of the end direction indicator may coincide with the longitudinal centre line may have a concave part, as seen from the rear end edge 5 and towards the front-end edge 4.

FIG. 2 shows an exploded view of the incontinence pad 1 in FIG. 1 a-c. and shows the first absorbent layer 12 and the second absorbent layer 13 where the first absorbent layer is located between the topsheet 6 and the second absorbent layer 13, and the second absorbent layer 13 is located between the first absorbent layer 12 and the backsheet 7. The central embossed shaping element 15 is in the second absorbent layer. The central embossed shaping element extend in the longitudinal direction along the longitudinal centerline (Lc). The first absorbent layer 12 has a greater extension than the second absorbent layer 13.

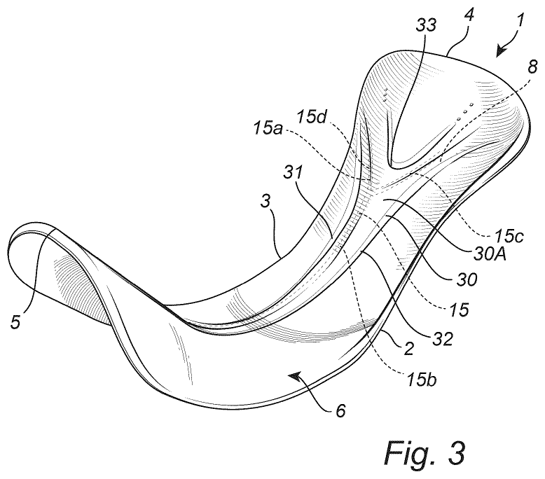

FIG. 3 illustrates the incontinence pad 1 from FIGS. 1 and 2 when being flexed and shaped as during use. The disclosed configuration of the shaping indicator 30 support and give the user a visual guidance how to wear the incontinence pad 1 so as to closely adopt to the female anatomy and the core shaping element allows the front half of the article to fit over and conform to the flattened forward portion of the female genitalia and to form a front bowl shape for acquiring the body liquids, while the rear half of the article aligns with and fit comfortable into the crease-like regions of the buttocks and perineum

The shaping indicator 30 is in FIG. 1-3 printed but the shaping indicator may also be formed by embossing or a combination of print and embossments.

### Thickness Measurement

The thickness of the absorbent layer is defined by means of a measuring foot with affixed load of 0.5 k Pa. The foot is lowered onto the absorbent layer at a rate of 13 mm/s. The thickness is read off at the digital thickness gauge/tester after 5 seconds when the measuring foot has touched the surface of the absorbent layer.

1. An absorbent article for a female user in the form of a sanitary napkin or incontinence pad having a first and a second longitudinal side edges and a front end edge and rear end edge and comprising a fluid pervious topsheet a backsheet and an absorbent core being located between the topsheetand the backsheet, the absorbent article has a longitudinal direction with a longitudinal centerline, a transverse direction with a transverse centerline and a thickness direction, and has in the longitudinal direction a front half and a rear half, wherein the absorbent article comprises a shaping indicator, visible from the topsheet, extending along a longitudinal centerline and being symmetric about the longitudinal centerline and asymmetric about the transversal centerline, the shaping indicator has a shaping indicator area, a longitudinal first side edge, a longitudinal second side edge, a transversal front end edge and a transversal rear end edge, the widest transversal width of the shaping indicator is in the front half and the narrowest transversal width of the shaping indicator is in the rear half, and the absorbent core comprising a core shaping element extending in the longitudinal direction, which in the thickness direction is located within the shaping indicator area.

2. The absorbent article according to claim 1, wherein the shaping indicator area is printed or embossed.

3. The absorbent article according to claim 1, wherein a circumference around the shaping indicator area is printed or embossed.

4. The absorbent article according to claim 1, wherein the core shaping element is provided by embossment(s), cut(s), area(s) with less density than surrounding area(s) or area(s) with substantially no absorbent material.

5. The absorbent article according to claim 1, wherein the transversal width of the shaping indicator in the rear half of the absorbent article is less than 50% of the transversal width of the absorbent article in the rear half.

6. The absorbent article according to claim 1, wherein the shaping indicator is applied at least on the topsheet.

7. The absorbent article according to claim 1, wherein maximum transversal width of the shaping indicator in the rear half is within the range of ±10% of the minimum width of the shaping indicator in the rear half.

8. The absorbent article according to claim 1, wherein the longitudinal first side edge and the longitudinal second side edge of the shaping indicator diverges in the direction from the rear end edge of the absorbent article towards the front end edge.

9. The absorbent article according to claim 1, wherein the longitudinal first side edge of the shaping indicator is concave as seen from the longitudinal centerline and the longitudinal second side edge of the shaping indicator is concave as seen from the longitudinal centerline.

10. The absorbent article according to claim 1, wherein the shaping indicator is in the form of a Y-shaped or modified Y-shaped indicator comprising a central I-shaped portion provided in the rear half of the absorbent article and a V-shaped or modified V-shaped portion provided in the front half of the absorbent article.

11. The absorbent article according to claim 1, wherein the transversal front end edge is concave as seen from the transversal centerline.

12. The absorbent article according to claim 1, wherein the transversal width of the shaping indicator closest to the rear end edge of the article is 0.5-20 mm and the transversal width of the shaping indicator closest to the front end edge is 40-70 mm.

13. The absorbent article according to claim 1, wherein the article has an intersection point between the longitudinal centerline and the transversal centerline, and an intersection point where a transversal outermost line in the front of the shaping indicator intersects with a first longitudinal outermost sideline, and an imaginary line between the intersection and the intersection, wherein an angle a between the imaginary line (L a) and the longitudinal centerline which is in the interval between 20° to 45°.

14. The absorbent article according to claim 1, wherein the absorbent core has a front part, a rear part and an intermediate part located between the front part and the rear part, wherein the widest transversal width of the absorbent core is in the front part.

15. The absorbent article according to claim 14, wherein a transition width between the front part and the intermediate part of the absorbent core is narrower than the transversal width of the rest of the absorbent core.

16. The absorbent article according to claim 1, wherein the absorbent comprising a first absorbent layer and a second absorbent layer and the first absorbent layer is located between the topsheet and the second absorbent layer, and the second absorbent layer is located between the first absorbent layer and the backsheet wherein the central embossed shaping element is in the second absorbent layer.

17. The absorbent article according to claim 16, wherein the second absorbent layer has higher density than the first absorbent layer, and the first absorbent layer optionally has a greater extension than the second absorbent layer.

18. The absorbent article according to claim 1, wherein the first and second longitudinal edges of the absorbent article are essentially straight and essentially parallel to each other.
